(19) United States
(12) Patent Application Publication
(10) Pub. No.: US 2012/0280055 A1
(43) Pub. Date: Nov. 8, 2012

(54) METHOD AND DISPENSER FOR CONTROLLED RELEASE OF SEMIOCHEMICALS

(75) Inventors: Rodney G. Schneidmiller, Greenacres, WA (US); Qing-He Zhang, Spokane Valley, WA (US)

(73) Assignee: STERLING INTERNATIONAL INC., Spokane, WA (US)

(21) Appl. No.: 13/452,744

(22) Filed: Apr. 20, 2012

Related U.S. Application Data
(60) Provisional application No. 61/477,521, filed on Apr. 20, 2011.

(51) Int. Cl. A61L 9/04; B65B 3/02

(52) U.S. Cl. 239/6; 239/58; 53/452

(57) ABSTRACT

A method and packages for releasing volatiles of a semiochemical at a controlled or pre-selected rate are disclosed. The package containing the semiochemical includes at least one portion that is permeable to the volatiles. In some embodiments, the package is formed as a stick pack, and includes a plurality of micro-perforations having characteristics, such as size, density, and shape, that are selected to achieve a desired release rate. In an embodiment, the package is formed from a plurality of laminae, and at least one of the laminae is formed from a material that is selected for its permeability to the semiochemical volatiles. In an embodiment, the package includes a portion that is peeled away to expose a layer that is permeable to the semiochemical volatiles.

### CROSS-REFERENCE TO RELATED APPLICATION

This application claims the benefit of U.S. Provisional Application No. 61/477,521, filed Apr. 20, 2011, the entire disclosures of which are hereby incorporated by reference herein.

### BACKGROUND

Insect traps are known in the art. For example, certain insect traps are targeted to flying insects such as wasps, yellow jackets, flies and the like. Other traps are targeted to agricultural and residential nuisances such as stink bugs, shield bugs, boxelder bugs, kudzu bugs, and the like. Insect traps will frequently use one or more semiochemicals such as pheromones, kairomones, synomones, and attractants, to improve the efficiency and efficacy of the trap. Semiochemicals may also be used in other applications. The term semiochemicals is defined herein to include any chemicals that operate to modify or affect the behavior of an insect. For example, repellant-type semiochemicals may be used to drive certain insects out of an area or to prevent or reduce insect ingress into an area, such as a building, or to repel particular insects from an individual or animal. It is contemplated that suitably situated semiochemicals may be employed to urge target insects in a particular direction, for example, agriculturally beneficial insects may be “herded” towards a particular crop.

Typically, the semiochemical vapors or volatiles are released gradually into the air, such that the volatiles form a plume or region having a relatively high concentration of the semiochemical. Volatilization is defined to be the process of converting a chemical substance from a liquid or solid state to a gaseous or vapor state. Volatile when used as a noun is defined to refer to the converted chemical in the gaseous or vapor state. Generally, a semiochemical will volatilize at a rate that depends on the volatility of the semiochemical, properties of any matrix or other substance combined with the semiochemical composition, environmental conditions, and/or the exposed area of the semiochemical.

Achieving a desired and predictable release rate of the volatiles is important in insect traps and in other applications where the gradual release of a volatilizable substance is desired. To optimize the performance of the semiochemical, a particular range of concentration of the semiochemical vapor may be desired. The efficacy of the semiochemical to perform its intended function may decrease if the concentration is too high or too low. Also, there may be aesthetic disadvantages if the semiochemical is released at too high a rate, for example, undesirable odors or the attraction of non-target insects into the vicinity.

It will also be appreciated that an insect trap typically is expected to continue trapping target insects for an extended period of time. If the trap relies on a semiochemical for its effectiveness, for example, an attractant, the rate of release of the semiochemical should be gradual such that the semiochemical is not too quickly exhausted.

Different methods have been used in attempts to control the rate of release of semiochemical volatiles. For example, a quantity of the semiochemical may be combined with or disposed in a porous substrate, e.g., plastic, ceramic, sponge, paper, or the like. In another example, the semiochemical may be partially enclosed in a non-porous wrapping or container such that only a relatively small portion of the semiochemical composition is exposed to the air. Common types of semiochemical dispensers for commercial and research purposes include rubber septa, polyethylene (PE) bags, PE-tubes, bubble caps, wicks, laminate plastics, and glass vials with openings of various sizes. However, it remains difficult to achieve a desired volatilization or release rate for a semiochemical that will remain relatively consistent over an extended period of time.

Also, certain prior art dispensers may be suitable for research use and/or for small to medium scales of commercial production (e.g., with manual or semi-automation loading). However, such systems are typically not suited to large scale commercial production. Therefore, new types of controlled-release semiochemical dispensers that can be manufactured via modern packing technology are strongly needed.

Stick packs, typically elongate tubular packaging that is sealed at both ends, have been around for years. Stick packs have more recently become popular in the packaging industry for foods, beverages, and medicines. A key objective in some stick pack technology is to maintain the freshness of the contents of the stick pack (liquids, creams, powders, etc.) until the contents are used. To this end, a packaging film is used comprising at least two layers, with the outer layer(s) typically made from a high density barrier material and one or more inner layers made of lower density material suitable for heat-sealing.

Disclosed here is a novel method and device to use the stick pack technology for the controlled release of volatiles, for example, volatiles of insect semiochemicals.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

A method and package for providing a controlled release of volatiles, for example, semiochemical volatiles for controlling the behavior of target insects, includes a package containing the semiochemical with means providing for a controlled release of the volatiles. A particular method includes forming a stick pack from a polymeric material and having means for permitting the desired volatiles to pass through the polymeric sheet. The means may comprise a permeable or semi-permeable panel that permits semiochemical to be released gradually. A cover, for example a peal-away layer of the panel, may enable the user to activate the release by uncovering a portion of the permeable panel. The means may include micro-perforations, which can be provided in a desired size, shape, density, depth, and the like to achieve a desired release rate. The means may include one or more layers of the polymeric sheet having permeability properties that permit the desired volatiles to pass through. The semiochemical is deposited into the stick pack, and the stick pack is closed, to retain the semiochemical therein.

In one embodiment, the polymeric sheet is formed from a plurality of laminae with preselected thickness to achieve the desired release rate.

In one embodiment, the semiochemical comprises a liquid or gel composition containing the semiochemical. In another embodiment, the semiochemical comprises a powder with particles having a characteristic dimension that is larger than a characteristic dimension of micro-perforations in the stick pack, such that the powder is retained in the stick pack. The semiochemical may be, for example, an insect repellant or an insect attractant for the target insect species.

In a package, the stick pack is formed from a polymeric sheet with means for permitting a semiochemical volatile to pass therethrough, and a quantity of semiochemical disposed and retained in the stick pack, wherein the stick pack is configured to achieve the desired release rate of volatiles.

The polymeric sheet may be constructed of multiple laminae, and include a plurality of micro-perforations and/or a lamina formed from a material that is permeable or semi-permeable to the semiochemical volatiles. The package may be sealed in an outer package for shipping and display. In one embodiment, the stick pack includes one or more peel-away portions that are removed by the end user to expose micro-apertures or permeable membranes in the stick pack, to initiate or adjust the rate of release of volatiles.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A shows a front view of a first embodiment of a semiochemical stick pack in accordance with the present invention;

FIG. 1B shows a side view of the semiochemical stick pack shown in FIG. 1A;

FIG. 1C shows an end view of the semiochemical stick pack shown in FIG. 1A;

FIG. 2A shows schematically a cross section of the semiochemical stick pack through section 2-2 in FIG. 1A, showing a solid particulate semiochemical composition therein;

FIG. 2B shows schematically a cross section of the semiochemical stick pack through section 2-2 in FIG. 1A, showing a liquid semiochemical composition therein;

FIG. 3 shows a fragmentary cross-sectional view of a sheet material for semiochemical stick packs in accordance with the present invention, wherein the various dimensions are exaggerated to illustrate aspects of the sheet material;

FIG. 4 shows schematically a system diagram for an apparatus for producing the semiochemical stick pack shown in FIG. 1A, packaged with a semiochemical;

FIG. 5 is a flow diagram illustrating an exemplary method for controlling the rate of release of volatiles of semiochemicals in accordance with the present invention;

FIG. 6 illustrates another embodiment of a multi-compartment semiochemical stick pack in accordance with the present invention;

FIG. 7 illustrates another embodiment of a semiochemical stick pack, having a window portion for the controlled release of volatiles;

FIGS. 8A and 8B illustrate a panel for forming another embodiment of a semiochemical stick pack in accordance with the present invention, wherein FIG. 8B is a sectional view through section 8B-8B in FIG. 8A;

FIG. 9 is a front view of the stick pack formed from the panel shown in

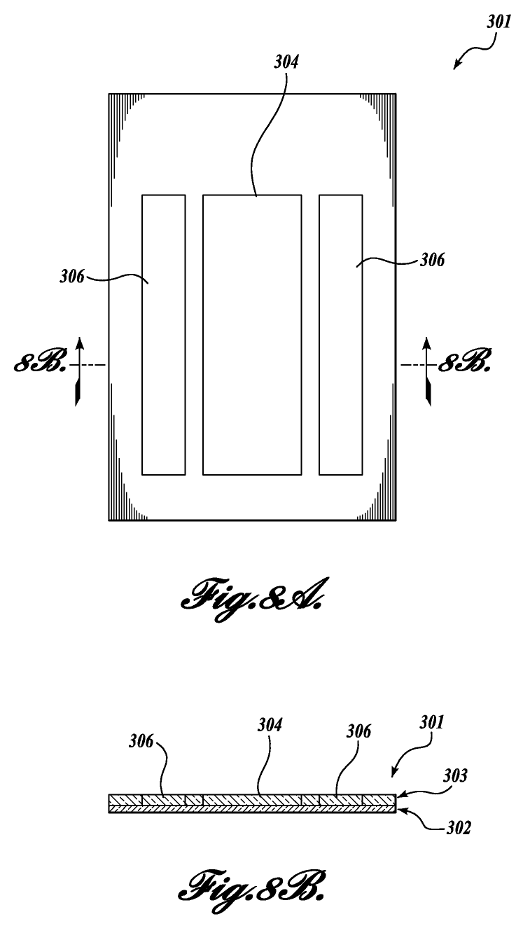

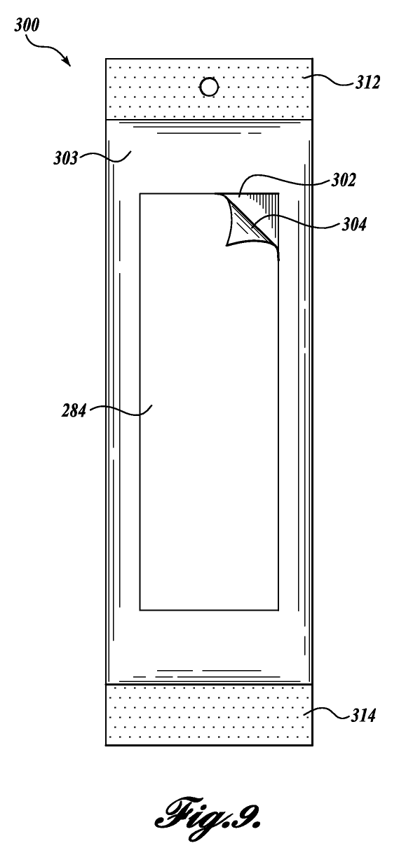

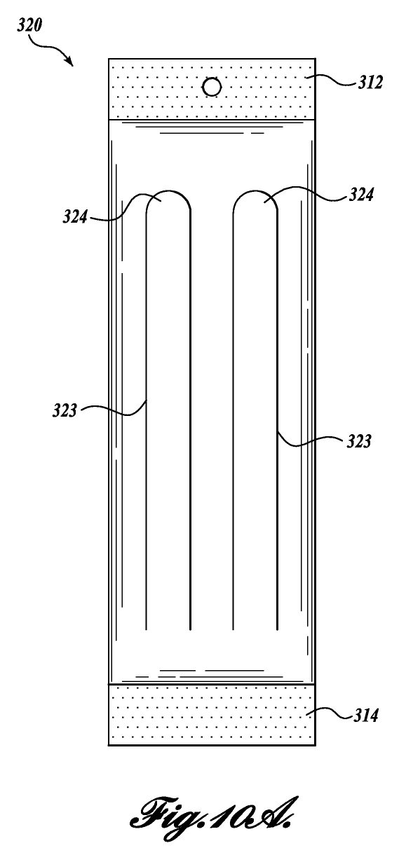

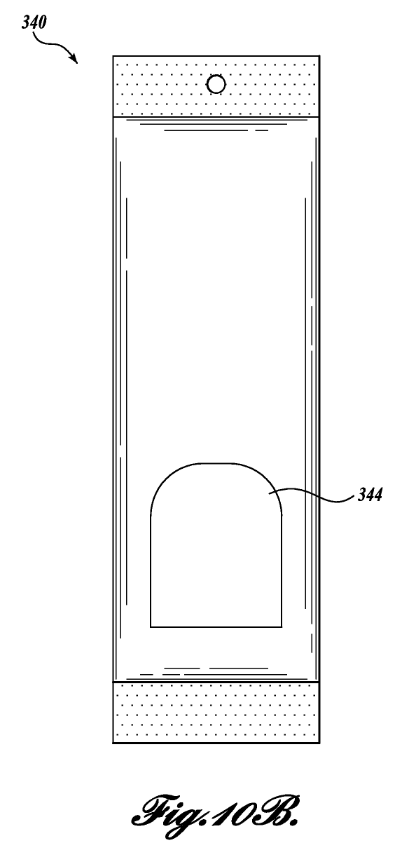

FIGS. 8A and 8B; and

FIGS. 10A and 10B show alternative designs for a stick pack similar to the stick pack shown in FIG. 9, with different window configurations.

### DETAILED DESCRIPTION

Particular embodiments of stick packs for the controlled release of volatiles in accordance with the present invention will now be described with reference to the figures, wherein like numbers indicate like parts. Although for clarity and ease of understanding the disclosed application is directed to means for controlling the release rate for semiochemical volatiles, it will be appreciated that the disclosed means are readily applicable to controlling the release rate of any volatile substance.

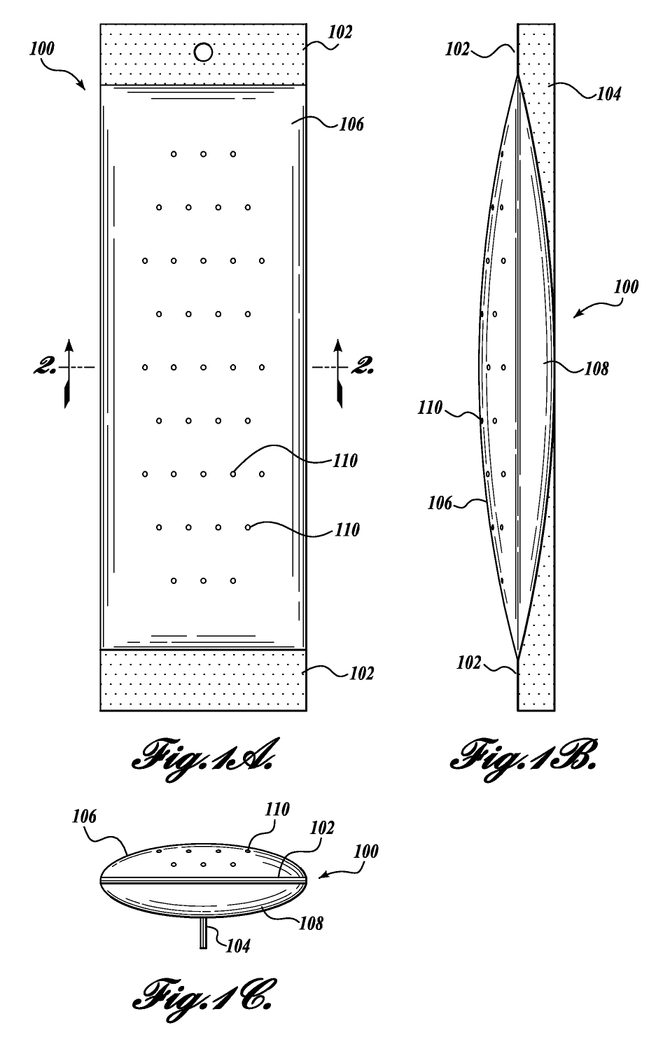

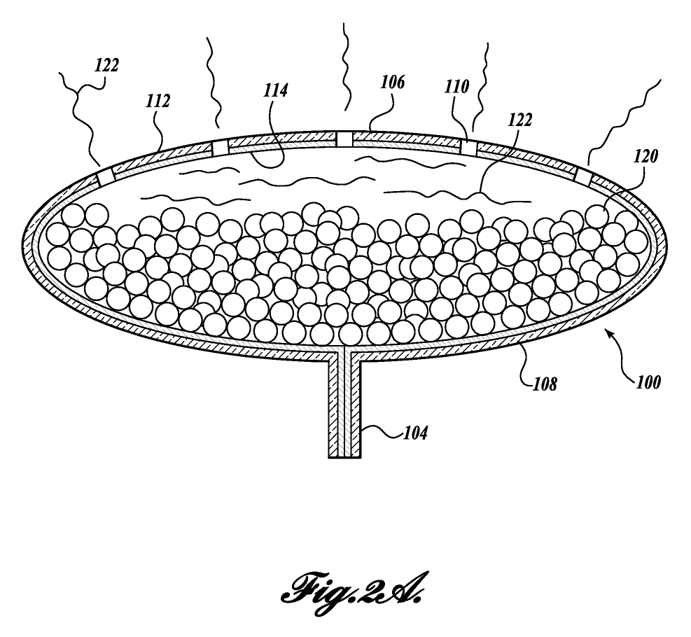

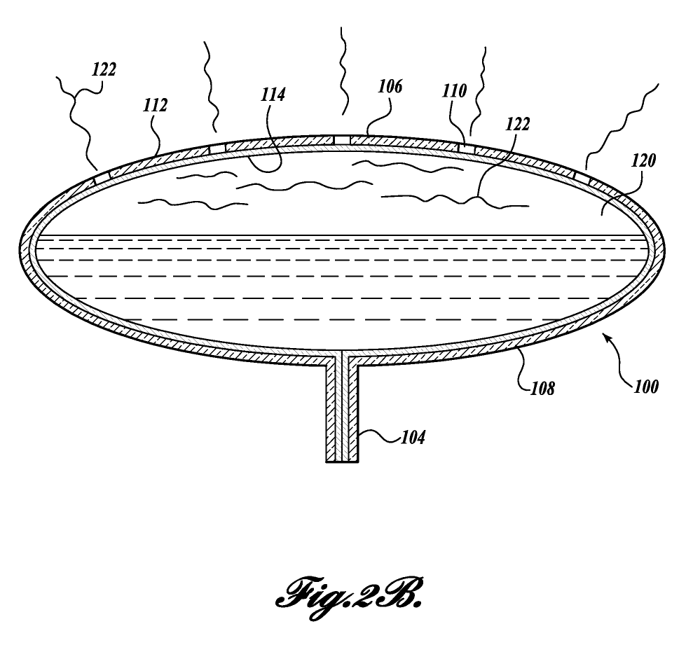

FIGS. 1A, 1B, and 1C illustrate front, side, and end views, respectively, of a sachet or stick pack 100 in accordance with the present invention. The stick pack 100 is a generally tubular structure formed from a sheet of material, preferably a polymeric sheet comprising multiple layers or laminae. The end portions 102 are sealed transversely, and a longitudinal sealed portion 104 closes the tubular structure, such that a volume is defined between the first and second ends 102.

As discussed below, the properties and configuration of the multiple layers for stick packs 100 cooperatively restrict and control the release rate of volatiles from the semiochemicals that are packaged in the stick pack 100. In particular, the designer may select the materials and certain characteristics of the layers used for the sheet of material to achieve a desired volatile release rate. For example, the layer material properties (e.g., the porosity of the material to the selected semiochemical volatiles), the thickness of the layers, the characteristics of optional apertures (e.g., number, density, size, depth, and shape).

In the embodiment of FIGS. 1A-1C, a front panel 106 of the stick pack 100 optionally includes a pattern of micro-perforations 110 that are sized and configured to achieve a desired release rate of volatiles, as discussed below. In the current embodiment, a back panel 108 portion of the stick pack 100 does not include any micro-perforations. However, it is contemplated that in some applications it will be desirable that the back panel 108 also include micro-perforations 110. In some applications the stick pack may be formed from a suitably permeable or porous material, wherein the permeability is sufficient to achieve a desired volatile release rate without the use of micro-perforations. Furthermore, a punched hole 101 or the like may be provided in one or both of the end portions 102 for hanging or otherwise attaching the stick pack 100 to an object, for example an insect trap (not shown).

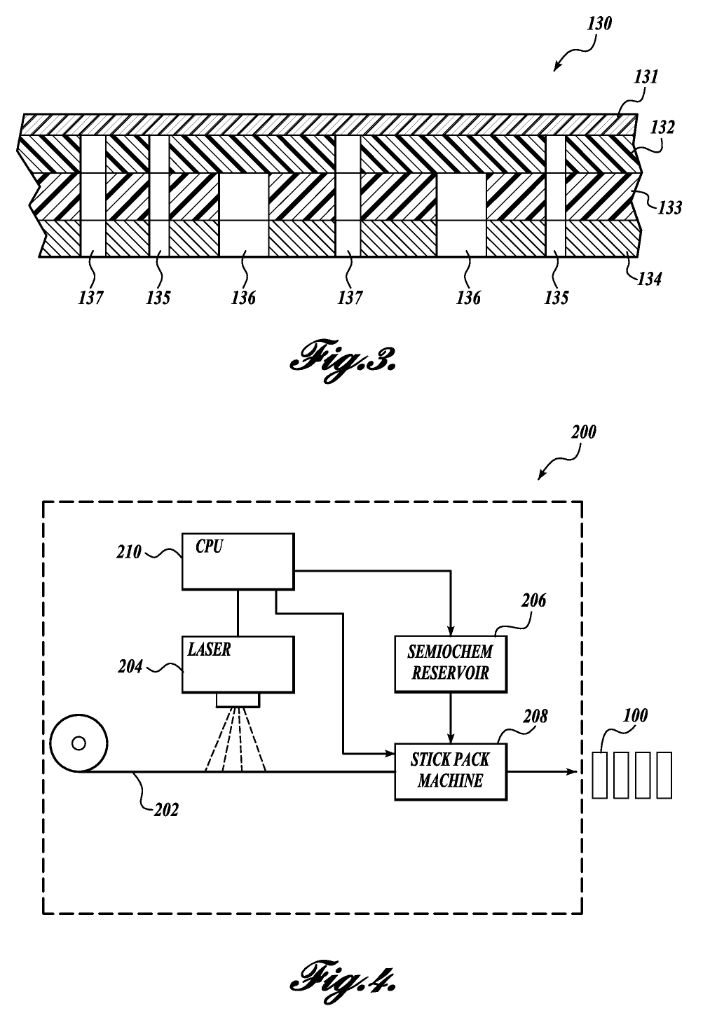

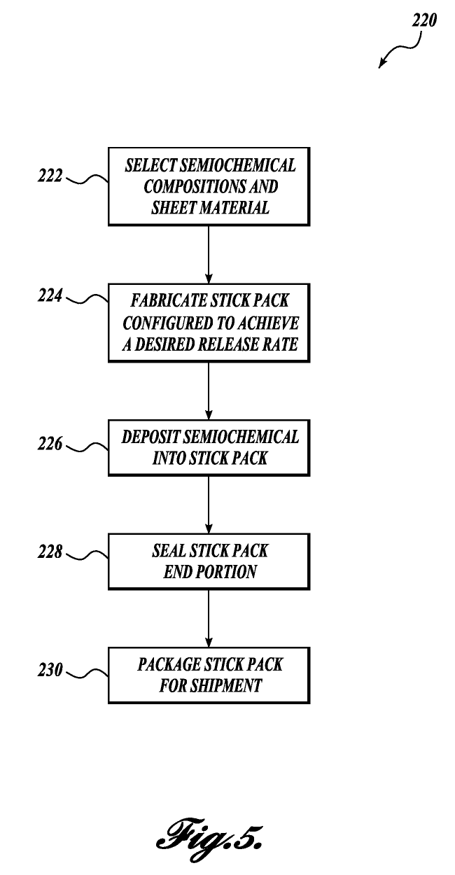

FIG. 2A illustrates a cross section of the stick pack 100 with the semiochemical composition 120 in the stick pack 100 illustrated generically. In this exemplary embodiment, the innermost layer 114 comprises a material having a relatively low density that is suitable for heat welding to form effective seals. The innermost layer 114 may also be selected for its permeability to the semiochemical volatiles 122. The outermost layer 112 is bonded or otherwise adhered to the innermost layer 114 and is formed of a relatively higher density material selected for its barrier functionality, mechanical strength, dimensional stability, and suitability for manipulation in a high speed stick pack machine (see, FIG. 4). The semiochemical composition 120 comprises one or more semiochemicals, and other components that may be desired, for example, to stabilize or otherwise affect the chemical or mechanical properties of the composition 120.

In FIG. 2A, the semiochemical composition 120 is illustrated in an idealized bead, powder, or particulate form having a characteristic size or dimension (e.g., diameter). Preferably, the micro-perforations 110 are sized to prevent the loss of the particulates therethrough. It is also contemplated that the semiochemical composition 120 may alternatively be in liquid form, incorporated into a gel, paste, or solid matrix, or absorbed into a porous medium such as a sponge or paper, for example. In liquid form, the semiochemical composition may be of relatively low viscosity, or a very viscous or viscoelastic material. The selected semiochemical volatilizes at the environmental conditions contemplated for its intended use. The quantity of semiochemical composition 120 may be such that the volume enclosed by the stick pack 100 is only partially filled by the composition 120. The remaining volume in the stick pack 100 may be partially or substantially filled with semiochemical vapors or volatiles 122. The volatiles 122 escape or are gradually released through the micro-perforations 110, and/or through any permeable layer defined by the stick pack 100.

In FIG. 2B, the semiochemical composition 120 is illustrated in an idealized liquid form. If the semiochemical composition 120 is in liquid form it is contemplated that the micro-perforations 110 will extend only through the outer layers 112, and the non-perforated inner layer 114 will therefore prevent any leakage of liquid semiochemical composition 120 therethrough. The inner layer 114, of course, is selected to permit a gradual release of semiochemical volatiles.

The rate of release of the volatiles 122 will depend in part on the characteristics of the micro-perforations 110. For example, the rate of release may depend on micro-perforation parameters such as (1) the number of perforations; (2) the size or distribution of sizes of the perforations; (3) the spacing and pattern of the perforations; (4) the shape of the perforations (e.g., elongate, star-shaped, circular); (5) the depth of the perforations (e.g., extending partially through the substrate); and (6) any blockage of the perforations. The designer and/or the user, therefore, have a number of parameters that may be used to control the rate of release of volatiles 122.

For example, the designer may select the size and number of micro-perforations 110 to accommodate a particular semiochemical or combination of semiochemicals 120 to achieve a desired release rate. A composition 120 having a semiochemical with a low volatility may require more and larger perforations than one with a semiochemical that is highly volatile. In another example, different configurations of micro-perforations 110 may be available, depending on the anticipated environmental conditions (e.g., temperature, humidity) for the expected use of the semiochemicals composition 120. For example, one configuration of micro-perforations in a semiochemical stick pack 100 may be suitable when lower temperatures are expected, and a different configuration may be suitable at higher temperatures. A family of semiochemical stick packs 100 may be made available to users, who will then select the particular stick pack 100 that suits their application. Optionally, a blocking element (not shown), for example, a strip of adhesive, a sleeve, or the like, may be provided to selectively block some portion of the micro-perforations 110, to selectively adjust the rate of release of volatiles 122, for example, to adjust for environmental conditions or to accommodate particular situations.

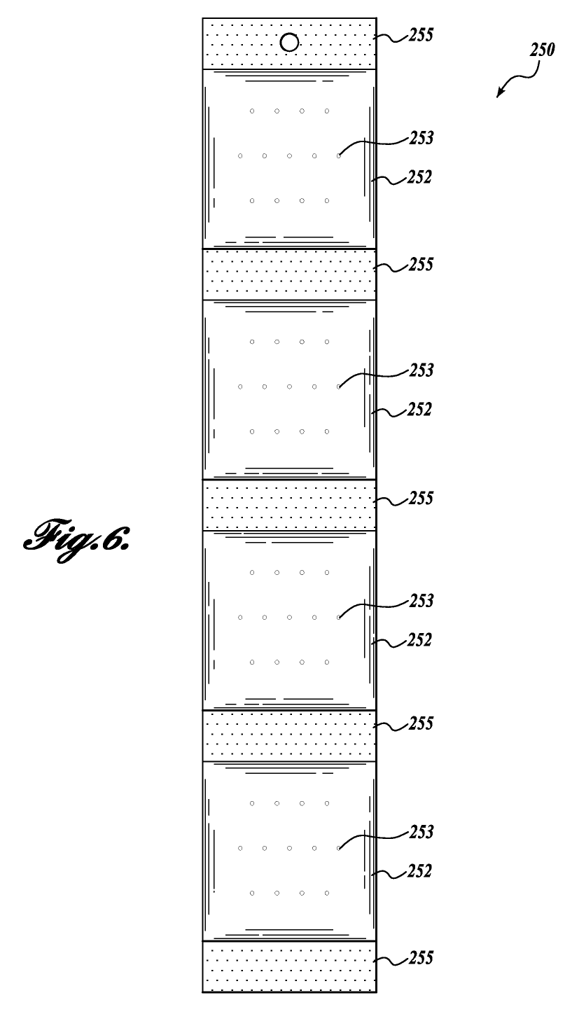

FIG. 3 illustrates an exemplary fragmentary cross section of a sheet 130 that may be used to form the stick pack 100. The sheet 130 includes one or more polymeric laminae, and may additionally include paper or foil laminae (barrier layer), for example. In this exemplary embodiment, the sheet 130 comprises four laminae 131, 132, 133, 134. An exemplary total thickness of the sheet 130 is in the range of 5.0 to 400.0 microns. In a current embodiment, the total thickness is between about 30.0 microns and 300.0 microns. The multiple laminae 131, 132, 133, 134 may be provided to produce a desired release rate of volatiles 122, and to achieve desired mechanical and manufacturability properties. For example, the material for the innermost lamina 131 may be selected, in part, for its ability to produce good and consistent longitudinal and end seals for the stick pack 100.

The material for one or more of the laminae 131, 132, 133, 134 may also be selected based on the permeability of the material to the semiochemical volatiles, providing an additional parameter to control the release rate of particular volatiles 122.

In FIG. 3, the micro-perforations have varying diameters and varying depths of penetration through the sheet 130. For example, micro-perforations 135 are relatively small in diameter and extend through the outer lamina 134 and all of the way to the inner lamina 131. If a solid semiochemical is to be used, for example, the micro-perforations 135 may alternatively extend through the inner lamina 131. Therefore, molecules of suitable size may escape from the stick pack 100 through the apertures 135. Micro-perforations 136, although relatively large in diameter, only extend through the two outermost laminae 133, 134. Therefore, only molecules that are permeable to the innermost laminae 131, 132 will readily escape through these micro-perforations 136. Micro-perforations 137 are of intermediate diameter, and extend through the three outermost laminae 132, 133, 134 in this exemplary embodiment.

Therefore, it will be appreciated that a stick pack 100 may be designed to contain a plurality of different semiochemicals in a mixture or agglomeration, and to provide different release rates for each of the different semiochemicals.

FIG. 4 illustrates a system 200 for producing a stick pack 100 containing one or more semiochemicals. The system 200 in this embodiment takes a roll of sheet material 202 and selectively directs a laser system 204 to produce a desired pattern of micro-perforations in or through the sheet material 202. Different commercial laser systems are suitable. For example, it is known in the packing industry to use CO₂ lasers, such as “sealed off” coherent CO₂ lasers. Such lasers are suitable for use to process paper, plastic film, and other flexible materials. By some accounts, the sealed off coherent CO₂ laser has become a tool of choice to process packaging materials due to its reliability, low cost, compact footprint, and high quality with respect to laser power and beam characteristics.

A reservoir 206 of the desired semiochemical composition provides product to a stick pack machine 208 that receives the sheet material 202 and forms the final stick pack 100 of semiochemical composition 120. The operation is controlled with a computer or stand-alone central processing unit (CPU) controller 210 that may be separate or integrated into the stick pack machine 208. The controller 210 is programmable to accommodate different sheet material 202 and semiochemicals 120, such that the system 200 may be operated to produce any number of different products.

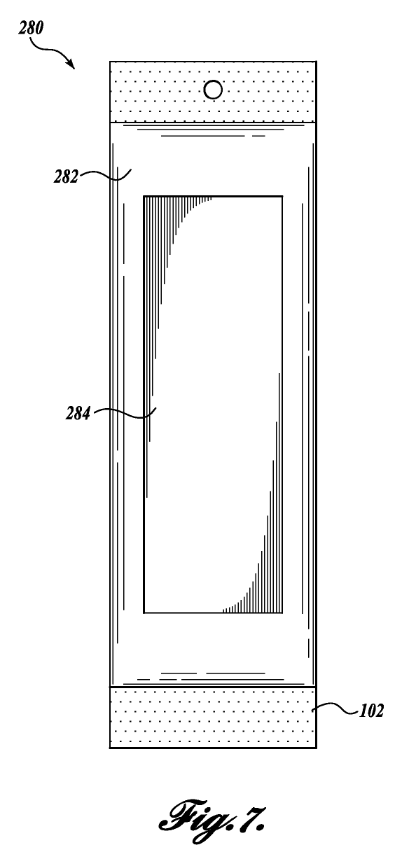

A simplified flow chart 220 of a method in accordance with the present invention is shown in FIG. 5. The user first selects 222 one or more semiochemicals and sheet material for a particular application. The semiochemicals are selected with reference to the target insect. For example, the semiochemicals may comprise one or more attractants for the brown marmorated stink bug. The selection of semiochemical(s) will include selection of the particular form and composition of the semiochemical, including any matrix material that may be useful for stabilizing or controlling the volatilization of the semiochemical. It may also be desirable to include semiochemicals that repel non-target insects. The composition may also include components to confer particular aesthetic aspects to the composition, such as color or scent. A composite sheet material for the stick pack package is also selected. The selection of the sheet material 202 may require consideration of the particular semiochemical composition selected. For example, the innermost lamina of the sheet material must be compatible with the semiochemical. One or more of the laminae may be selected for their permeability with respect to one or more of the semiochemicals.

The packaging for the stick pack 100 is fabricated 224, configured for the desired release rate of the volatiles, for example, with micro-perforations and/or selected permeability properties. The selected semiochemical(s) are deposited into the packaging or onto the sheet prior to sealing the package 226. The stick pack ends and longitudinal seam are sealed 228. The stick pack 100 may then be sealed in an outer package 230, for example, a foil pack or a plastic package, which is suitable for shipping and display. The sealed outer package inhibits the release of the volatiles prior to use. As an alternative or in addition, it is contemplated that a removable adhesive strip (not shown) may be placed over the micro-perforations and removed prior to use.

Although the above described stick pack 100 is formed with a single compartment for the semiochemical composition 120, it is contemplated that the stick pack may be formed with multiple compartments. FIG. 6 illustrates an exemplary multi-compartment stick pack 250. In this embodiment, four separate compartments 252 are defined in the stick pack 250, each separate compartment delineated by sealed ends 255. Although four compartments are shown, more or fewer compartments are also clearly contemplated. The individual compartments may all be of similar or identical physical characteristics, e.g., micro-perforation 253 size, pattern, and depth. For example, separate adhesive strips (not shown) may be applied over the micro-perforations 253 in each compartment 252, such that the compartments 252 may be individually opened for releasing volatiles. This gives a user the option to open multiple compartments 252 initially to increase the rate of release of semiochemicals, or to open each compartment 252 only after the previous compartment semiochemical has been exhausted or lost its effectiveness.

Alternatively, the compartments 252 may be configured differently, for example, to accommodate different semiochemical compositions 120. The multi-compartment stick pack 250 may therefore be readily designed to accommodate different semiochemicals, with the micro-perforations in each compartment 252 tailored to produce a desired rate of release of volatiles for each semiochemical. As discussed above, a punched hole may be included for hanging or otherwise attaching the stick pack 250 to a device.

Another exemplary embodiment of a semiochemical stick pack 280 in accordance with the present invention is illustrated in FIG. 7. The stick pack 280 sachet is formed from a sheet material having at least an outermost lamina 282 as a barrier layer and an innermost lamina 284 as a sealing and releasing layer. This embodiment is similar to the stick pack 100 described above, except that rather than (or in addition to) a plurality of micro-perforations, windows are formed in the outermost lamina 282, defining an opening or “window” in the sachet that exposes the innermost lamina 284. The innermost lamina 284 may be permeable to the semiochemical volatiles to permit a gradual release rate and/or may include micro-perforations (not shown) to further control the release rate. The innermost lamina 284 is therefore exposed for release of volatiles. A packaging or other external barrier (not shown) to prevent or mitigate release of the semiochemical before deployment of the stick pack 280 storage before use could be provided. The packaging and stick pack are configured to maintain the integrity of the semiochemical contents over time, e.g., during shipment and storage, such that the semiochemical product will produce the desired release rate and retain its efficacy when the stick pack is deployed.

Another exemplary embodiment of a semiochemical stick pack 300 in accordance with the present invention is illustrated in FIGS. 8A, 8B, and 9. FIG. 8A is a plan view of a portion of a sheet of material 301 for producing a single stick pack 300. It will be appreciated that the sheet of material would typically be configured on a continuous roll (not shown), and may include templates or room for multiple stick packs 300 across the width of the roll. FIG. 8B is a cross-sectional view of the unit template shown in FIG. 8A, with the depth dimension exaggerated for clarity.

In this embodiment, the inner layer 302 shown on the bottom in FIG. 8B is configured to define the inner lamina of the stick pack 300, and is adhered to an outermost layer 303. The outermost layer 303 includes one or more peel-away portions 304, 306 that are configured to be removed just prior to use, to open “windows” exposing a portion of the inner layer 302. The inner layer 302 may comprises a plurality of laminae, perhaps including micro-perforations as shown in FIG. 3, or may be a single layer without micro-perforations, and having a permeability to the semiochemical to provide the desired release rate.

It is contemplated that the peel-away portions 304, 306 may be produced using different methods, as are known in the art. In an exemplary method the peel-away portions 304, 306 are created or defined by leaving a selected window portion of the inner layer un-laminated during the sheet-making process, and laser scoring or cutting the outer layer 303, without cutting the inner layer 302. The peal-away operation may be carried out as part of the film-making process, i.e., before the stick pack is formed, or may be left for the end-user to perform, for example immediately before use.

FIG. 9 shows a front view of the stick pack 300, fully assembled and therefore containing the desired semiochemical. End seals 312, 314 close the stick pack 300 at the top and bottom ends, and a longitudinal seal 314 closes the lateral edges to define the tube structure. The first peel-away portion 304 is shown partially removed to expose a portion of the inner layer 302.

This packaging arrangement provides the end-user with great control and flexibility in controlling the release rate of the semiochemical contained therein, by allowing the end-user to determine how much of the peel-away portion 304 to peal down, and similarly how much of the options back side peal-away portions 306. For simplicity in manufacturing, in a current embodiment the peal-away portions are formed only on the front side of the stick pack.

FIGS. 10A and 10B illustrate other embodiments of stick packs 320 and 340, respectively. The stick pack 320 includes two peal-away portions 324 defined by cuts or score lines 323. The score lines 323 are not closed, and therefore the peal-away portions 324 will generally remain attached to the stick pack 320. The multiple peal-away portions 324 allow an end-user to control the rate of release of volatiles from the stick pack 320 by pealing one or both of the peal-away portions 324 and/or by electing how far to pull the peal-away portion(s) 324 down the stick pack 320. Of course, more than two peal-away portions may used. In the embodiment in FIG. 10B, the stick pack 340 has a peal-away portion 344 that is much shorter than the length of the stick pack 340. Thus, for example, the user may more precisely control the location that volatiles are released from the stick pack 340.

The stick packs in accordance with the present invention may alternatively be used to release a very large amount of insect pheromone in the field, for example to disrupt the normal mating behavior of target insects (a pest control approach called “mating disruption”.

The stick packs in accordance with the present invention provide a mechanism for very precisely controlling the release rate of semiochemicals contained in the stick pack. In exemplary uses, the stick packs may be used in insect traps to lure the target insect into the trap with an attractant.

Alternatively, stick packs containing repellant semiochemicals may be distributed about a particular perimeter to drive a target species away from a region, and/or to discourage the target insect from entering the region. For example, the stick packs may be placed around the points of entry into a building, or around a tent or other portable shelter.

It is believed that all or most insects use semiochemicals that affect the behaviors of other individuals, for example other members of the insect species or individuals in other species. Pheromones provide intra-species signals that aid in finding mates, food and habitat resources, warning of enemies, and avoiding competition. Allomones and kairomones provide interspecies signals that provide similar functions. The goals of using semiochemicals in insect management are typically to monitor populations, and/or to alter insect behavior, for example to reduce target pest insect populations. Semiochemicals generally have the benefits of being highly targeted, relatively nontoxic, nonpersistent and environmentally safe, and difficult for insects to develop resistance against.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for controlling the rate of release of volatiles of a semiochemical comprising:
forming a stick pack comprising a polymeric sheet having means for permitting semiochemical volatiles to pass through the polymeric sheet;
providing a quantity of semiochemical into the stick pack; and
sealing an end portion of the stick pack such that the quantity of semiochemical is retained in the stick pack;
wherein the stick pack is configured to prevent the quantity of semiochemical from escaping from the stick pack in a non-volatilized state and to achieve a desired release rate of volatiles of the semiochemical from the stick pack.

2. The method of claim 1, wherein the polymeric sheet comprises a plurality of laminae.

3. The method of claim 2, wherein an innermost lamina of the plurality of laminae is semi-permeable to volatiles of the quantity of semiochemical.

4. The method of claim 1, wherein the means for permitting semiochemical volatiles to pass through the polymeric sheet comprises an inner lamina that is permeable to the semiochemical vapors and an outer lamina that is configured to peal away from the inner lamina.

5. The method of claim 1, wherein the means for permitting semiochemical volatiles to pass through the polymeric sheet comprises a plurality of micro-perforations.

6. The method of claim 4, wherein the polymeric sheet further comprises an innermost lamina and wherein at least some of the plurality of micro-perforations do not penetrate the innermost lamina.

7. The method of claim 1, wherein the quantity of semiochemical is one of an insect attractant and an insect repellant.

8. The method of claim 1, wherein the quantity of semiochemical comprises a liquid, paste, or gel composition containing the quantity of semiochemical.

9. A package containing a semiochemical comprising:
a stick pack comprising a polymeric sheet having a means for permitting semiochemical volatiles to pass through the polymeric sheet; and
a quantity of semiochemical disposed in the stick pack, wherein the quantity of semiochemical volatilizes to generate semiochemical volatiles;
wherein the stick pack is configured to achieve a desired rate of release of the semiochemical volatiles though the stick pack.

10. The package of claim 9, wherein the means for permitting the semiochemical volatiles to pass through the polymeric sheet comprises an inner lamina that is permeable to the semiochemical volatiles and an outer lamina that is configured to peal away from the inner lamina.

11. The package of claim 9, wherein the means for permitting the semiochemical volatiles to pass through the polymeric sheet comprises a plurality of micro-perforations.

12. The package of claim 11, wherein at least some of the plurality of micro-perforations do not extend all the way through the polymeric sheet.

13. The package claim 11, wherein the polymeric sheet comprises a plurality of laminae.

14. A method of forming a package for releasing volatiles of a semiochemical comprising:
forming a stick pack comprising a polymeric sheet having an inner layer that is permeable to volatiles of the semiochemical and an outer layer that is substantially impermeable to volatiles of the semiochemical, wherein the outer layer is removably affixed to the inner layer;
cutting a closed contour through the outer layer to define a peel-away portion;
depositing a quantity of the semiochemical into the stick pack, wherein the semiochemical will gradually volatilize during use; and
sealing an end portion of the stick pack.

15. The method of claim 14, wherein the inner layer comprises a panel having a plurality of micro-perforations.

16. The method of claim 15, wherein the inner layer comprises a plurality of laminae.

17. The method of claim 15, wherein at least some of the plurality of micro-perforations do not penetrate an innermost lamina of the plurality of laminae.

18. The method of claim 14, wherein the step of cutting the closed contour is accomplished by die cutting or laser cutting.

19. The method of claim 14, wherein the quantity of semiochemical comprises one of an insect attractant and an insect repellant.

20. The method of claim 14, wherein the quantity of semiochemical comprises a mixture of semiochemicals.

21. A stick pack for controlling a rate of release of volatiles from the stick pack comprising:
a polymeric sheet having a plurality of micro-perforations, the stick pack defining a volume; and
a quantity of semiochemical disposed in the volume;
wherein the plurality of micro-perforations are sized and configured to prevent the quantity of semiochemical from escaping from the stick pack in a non-volatilized state and to achieve a desired release rate of volatilized semiochemical from the stick pack.

22. The stick pack of claim 21, wherein the polymeric sheet comprises a plurality of laminae.

23. The stick pack of claim 22, wherein an innermost lamina of the plurality of laminae is semi-permeable to volatiles of the semiochemical.

24. The stick pack of claim 23, wherein the semiochemical is targeted to a particular insect species, and comprises one of an attractant and a repellant.
